(19) United States
(12) Patent Application Publication
(10) Pub. No.: US 2002/0134373 A1
(43) Pub. Date: Sep. 26, 2002

(54) Aerosol generation using sterile multiple dose containers

(76) Inventor: Igor Gonda, San Francisco, CA (US)

Correspondence Address: Karl Bozicevic, BOZICEVIC, FIELD & FRANCIS LLP, 200 Middlefield Road, Suite 200, Menlo Park, CA, 94025, US

(21) Appl. No.: 09/813,451

(22) Filed: Mar. 20, 2001

(51) Int. Cl. A61M 11/00; B05B 7/00; A62B 7/00; A62B 9/00; A61M 15/00; A61M 16/00; A62B 18/00

(52) U.S. Cl. 128/200.14; 128/200.22; 128/200.19; 128/200.24

(57) ABSTRACT

A system for generating aerosol is disclosed, which system comprises a multiple-dose container, a device which provides the means for aerosol generation and delivery of an aerosol and a disposable package comprised of a single-dose container with a means for drug intake and which can be loaded into the device and filled with drug from the multiple-dose container, and an aerosol generating nozzle material. In one embodiment, the single-dose container of the disposable package comprises a needle which is used to penetrate a self-sealing, re-sealable area on a multiple-dose container holding drug under positive pressure. The drug leaves the multiple-dose container and enters the single-dose container via an open channel in the needle while preserving the sterility of the liquid in the multiple dose container. In another embodiment, both the multiple-dose container and a sterile single-dose container have self-sealing re-sealable areas thereon and drug can be transferred from the multiple-dose container to the single-dose container via syringe or an interconnecting needle which punctures the self-sealing, re-sealable area on both containers. This will enable preservation of sterility of the drug fluid both in the single dose and multiple dose containers.

### FIELD OF THE INVENTION

The field of invention relates to devices and methods of aerosol generation. More particularly, the invention relates to devices and methods of aerosol generation using sterile multiple-dose, refillable containers for delivery of an aerosol to a patient.

### BACKGROUND OF THE INVENTION

Delivery of respiratory medications by aerosol generation devices has long been the preferred method of intrapulmonary delivery for reasons of efficacy and minimization of side effects. Such aerosol generating devices generate an aerosol comprised of a pharmaceutically active drug and then facilitate the delivery of the aerosol to the patient by acting as a conduit through which the aerosol travels to the target site of the patient. The devices with which inhaled medications are administered have undergone a prolonged evolution. However, although this evolution has produced several safe and practical devices for aerosol delivery, it is still far from achieving an “ideal” device.

In most instances, these devices are supplied to the patient pre-loaded with the preferred drug, i.e., they are manufactured with the drug to be inhaled already loaded inside the device. However, significant problems exist with these devices as a consequence of their pre-loaded state. In such instances, the patient inhales or otherwise uses the drug in the device some time after it has been loaded into the device, often times a substantially prolonged period of time has passed since the drug was loaded into the device. Also, the type and quantity of drug to be inhaled by the patient is fixed at the manufacturing site, thus precluding such changes during the course of the drug therapy.

One significant problem with these pre-loaded devices involves development of specialized manufacturing methods and drug containers that provide the means of ensuring sterility during the manufacture and maintaining sterility of the drug formulation after it is loaded inside the aerosol generating device. This is particularly important in the light of the latest guidance promulgated by the U.S. Food and Drug Administration (FDA) (21 CFR Part 200, Docket no. 96N-0048, RIN 0910-AA88, Federal Register Vol. 65, no. 103, 2000, pp. 34082-34089) which requires that all aqueous solutions for inhalation be sterile and preservative free. There are important reasons for this FDA mandate. Non-sterile, aqueous solutions may allow for microbial growth. Accordingly, a small number of microorganisms in the solution initially could multiply to larger numbers before the patient uses the drug. Consequently, the microbial growth could spoil the drug solution before patient use, thus rendering the drug treatment ineffective. More importantly, patients who may have compromised lungs or immune systems could be adversely affected by the microbial growth. Even in circumstances where preservative is used in an inhalation formulation (e.g., outside U.S.A.), the manufacture and maintenance of sterility are desirable because microorganisms that are resistant to the preservative can emerge.

Thus, there is a need for an aerosol generation and delivery device which does not require that the drug be pre-loaded into the device at the manufacturing site in a sterile container suitable for multiple use, but could still generate and deliver an effective amount of a drug to a patient. On the other hand, the manufacture of sterile filled multiple dose containers for uses such as parenteral drug solutions is well developed. However, these containers are not suitable to be used directly in devices for aerosol generation because, during the use, the contents of the multiple dose container could get contaminated with microorganisms.

The present invention endeavors to address and solve these and other problems associated with the generation and delivery of sterile aerosolized drug to a patient.

### SUMMARY OF THE INVENTION

A system for generating a sterile aerosol is disclosed, which system comprises a multiple-dose container, a device which provides the means for aerosol generation and delivery of an aerosol and a disposable package comprised of a single-dose container with a means for drug intake and which can be loaded into the device and filled with drug or liquid for drug reconstitution from the multiple-dose container. The system allows for the use of a wide range of single-dose containers which can be filled from a multiple-dose container and then loaded into a device and aerosolized. The system also enables the use of a wide range of multiple dose containers that do not need to be specially developed for any one particular route of administration. Thus, for example conventional multiple dose ampoules, vials and bottles that are used currently for storage of sterile parenteral liquids can be used in conjunction with the current invention.

In one embodiment, the single-dose container of the disposable package comprises a needle which is used to penetrate a self-sealing, re-sealable area on a multiple-dose container holding drug or liquid for drug reconstitution under positive pressure. The contents leave the multiple-dose container and enter the single-dose container via an open channel in the needle. In another embodiment, both the multiple-dose container and single-dose container have self-sealing re-sealable areas thereon and drug or liquid for drug reconstitution can be transferred from the multiple-dose container to the single-dose container via syringe or an interconnecting needle which punctures the self-sealing, re-sealable area on both containers. The single dose container may have been previously sterilized so that when the sterile liquid is transferred to it aseptically as described above, the solution in the single dose container will be sterile and suitable for prolonged storage without the risk of microbial growth in it. Alternatively, the single dose container with the drug contained in it could be sterilized. In some instances, the single dose container may be used immediately after filling it, in which case it may not be as critical to assure the sterility of its contents,

An aspect of the invention is a multiple-component system which allows for the sterile transfer of drug or liquid for drug reconstitution from a multiple-dose container to a single-dose container for aerosolization as well as a method which uses such a system to transfer drug from the multi-dose container to a single dose container and thereafter create an aerosol.

Another aspect of the invention is a single-dose container having reduced pressure inside and having a hollow needle extending therefrom, the single-dose container having a breakable seal which maintains reduced pressure in the container until the needle is placed in a liquid formulation and the seal is broken, allowing the pressure in the liquid to move into the reduced pressure of the single-dose container.

Yet another aspect of the invention is a single-dose container as described above, further comprising a porous material which aerosolizes the liquid formulation forced through the material.

A further advantage of this invention is that drugs that are unstable in liquid form could be kept as solids (e.g., prepared by sterile freeze-drying) in the single-dose or multiple-dose containers. A sterile liquid such as USP Water for Inhalation, sterile saline for inhalation, etc., could be introduced via the re-sealable area of the single-dose or multiple-dose container to dissolve the solid, prior to use.

Another advantage of this system is that sterile drug transfer is quickly and efficiently obtained while maintaining the sterile integrity of the drug remaining in the multiple-dose container.

A feature of the invention is a single-dose container with a re-sealable, self-sealing area thereon.

An aspect of the invention is a single-dose container comprised of a formulation designed for delivering by inhalation, which single-dose container comprises a portion, area or wall which is a re-sealable area which self-seals after being punctured by an injection needle used to fill the single container, which single dose container is preferably further comprised of a porous material.

Another feature is a single-dose container with a conduit attached thereto.

These and other objects, advantages, and features of the invention will become apparent to those persons skilled in the art upon reading the details of the system and specific components as more fully described below.

### BRIEF DESCRIPTION OF THE DRAWINGS

FIG. 1 is a cross-sectional, schematic view of an embodiment of a single-dose container of the invention having a re-sealable area thereon.

FIG. 2 is a cross-sectional schematic view of a syringe in fluid communication with the contents of a multiple-dose container.

FIG. 3 is a cross-sectional schematic view of an embodiment of a single-dose container of the invention having a re-sealable area thereon, an interconnecting needle and a multiple-dose container.

FIG. 4 is a cross-sectional, schematic view of an embodiment of a single-dose container of the invention having a tubular member extending through a wall of the single-dose container.

FIG. 5 is a cross-sectional, schematic view of an embodiment of the invention showing an offset porous material and re-sealable area.

FIG. 6 is a cross-sectional, schematic view of an embodiment of the invention showing collapsible walls of a single-dose container.

FIG. 7 is a cross-sectional, schematic view of an embodiment of a single-dose container of the invention having a tubular member extending through a wall of the single-dose container in fluid communication with the contents of a multiple-dose container.

### DETAILED DESCRIPTION OF PREFERRED EMBODIMENTS

Before the present systems, devices and methods are described, it is to be understood that this invention is not limited to particular components and steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a drug” includes a plurality of such drugs cells and reference to “the needle” includes reference to one or more needles and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The terms “package” “container” and “disposable package” are used interchangeably herein and shall be interpreted to mean any receptacle , i.e., single-dose container, or two or more single-dose containers linked together by an interconnecting means wherein each single-dose container is adapted for being loaded into a device so that its contents can be aerosolized. The container will preferably include a re-sealable portion making it possible to aseptically add drug. The re-sealable portion may be comprised of rubber, a polymer or other suitable material which can be pierced by a needle and will self reseal when the needle is withdrawn. The container is preferably comprised of a porous material (as defined herein) or other type(s) of orifices suitable for aerosolization and may be collapsible (as described herein) making it possible to force the contents of the single-dose container out through a suitable orifice such as the orifices of a porous material. A single-dose container may include an opening covered by a porous material or an area with pores therein and channels which provide for fluid connection from the container to a porous material preferably not positioned directly over the container. The structural integrity of each container is designed such that fluid is forced through the porous material (without rupturing the container) in a manner such that the contents are aerosolized.

The contents of each container preferably consist essentially of a liquid, flowable formulation which includes a pharmaceutically active drug of any type and preferably a carrier. If the drug is not liquid or of a sufficiently low viscosity (to allow the drug to be aerosolized) carrier may be added to dissolve or suspend dry drug. The drug (and carrier if present) are preferably used without any additional material such as preservatives which might affect the patient. The formulation is a liquid, flowable formulation with a sufficiently low viscosity that can be readily aerosolized and is more preferably a flowable, liquid formulation consisting essentially of a pharmaceutically active drug dissolved or dispersed in an excipient carrier e.g. water, ethanol and/or mixture theory.

The term “multiple-dose container” shall be interpreted to mean any receptacle, container, vessel, bottle, vial, flask, ampoule, bag, pouch or the like used for holding and/or storing a sterile drug solution, sterile drug suspension, sterile powder to be reconstituted into a sterile liquid preparation for aerosolization or a sterile liquid such as USP Water for Inhalation, sterile saline or the like for reconstitution of a solid drug retained in a single-dose container. The multiple-dose container is comprised of a self-sealing, re-sealable area. The multiple-dose container can be disposable or refillable.

The term “respiratory drug” shall be interpreted to mean any pharmaceutically effective compound used in the treatment of any respiratory disease and in particular the treatment of diseases such as asthma, bronchitis, emphysema and cystic fibrosis. Useful “respiratory drugs” include those which are listed within the Physician's Desk Reference (most recent edition). Such drugs include beta adrenergics which include bronchodilators including albuterol, isoproterenol sulfate, metaproterenol sulfate, terbutaline sulfate, pirbuterol acetate and salmeterol formotorol; steroids including beclomethasone dipropionate, flunisolide, fluticasone, budesonide and triamcinolone acetonide. Anti-inflammatory drugs used in connection with the treatment of respiratory diseases include steroids such as beclomethasone dipropionate, triamcinolone acetonide, flunisolide and fluticasone. Other anti-inflammatory drugs include cromoglycates such as cromolyn sodium. Other respiratory drugs which would qualify as bronchodilators include anticholenergics including ipratropium bromide. The drugs can be also used to treat respiratory infections such as aminoglycoside antibiotics and colistin, or to clear thick mucus in cystic fibrosis such as recombinant human deoxyribonuclease, acetylcysteine etc. The term also includes drugs which have systemic effects such as proteins, peptides, nucleotide sequences (e.g., DNA in a vector), antibodies, receptors, vaccines, small molecules for treatment of systemic problems and the like. The present invention is intended to encompass the free acids, free bases, salts, amines and various hydrate forms including semi-hydrate forms of such respiratory drugs and is particularly directed towards pharmaceutically acceptable formulations of such drugs which are formulated in combination with pharmaceutically acceptable excipient materials generally known to those skilled in the art—preferably without other additives such as preservatives. Preferred drug formulations do not include additional components which have a significant effect on the overall formulation such as preservatives. Thus preferred formulations consist essentially of pharmaceutically active drug and a pharmaceutically acceptable carrier (e.g., water and/or ethanol). However, if a drug is liquid without an excipient the formulation may consist essentially of the drug which has a sufficiently low viscosity that it can be aerosolized using a dispenser of the present invention.

The term “drug” shall include “respiratory drug” as well as other types of drugs such as systemically effective drugs. The term is intended to encompass the presently available pharmaceutically active drugs used therapeutically and to further encompass to be developed therapeutically effective drugs which can be administered by the intrapulmonary route. The drug may be a protein such as EPO or insulin and, in particular, recombinantly produced human EPO or insulin. The insulin may be an insulin analog which may be a monomeric insulin such as insulin lyspro. Preferred drugs or insulin are those which can be effectively administered by inhalation and/or injection, but which are not effective or are less effective when administered orally. Human recombinant insulin and morphine are examples of systemic drugs which can be administered from the devices of the invention.

The terms “formulation” and “liquid formulation” and the like are used interchangeably herein to describe any drug as described above by itself or with a pharmaceutically acceptable carrier in any form, including a dry powder, but preferably in a flowable liquid form. Such formulations are preferably solutions, e.g. aqueous solutions, ethanolic solutions, aqueous/ethanolic solutions, saline solutions and colloidal suspensions.

The term “respiratory disease” shall be interpreted to mean any pulmonary disease or impairment of lung function. Such diseases include restrictive and obstructive disease and diseases such as emphysema which involve abnormal distension of the lung frequently accompanied by impairment of heart action. Restrictive diseases tend to limit the total volume of air that a patient is able to exchange through inspiration and expiration. Restrictive disease, such as can be present in certain types of fibrotic processes, can therefore be detected by reduced FVC indices. Obstructive disease, such as is present in patients with asthma, tends not to affect the total volume of air exchangeable through inspiration and expiration but rather the amount of time required for forced exhalation of air. In particular, the FEV₁ is markedly reduced in patients with acute asthma symptoms. More specifically, the FEV₁, when taken as a ratio of FVC (i.e. FEV₁ divided by FVC), is markedly reduced in patients with acute asthma. In addition to increasing the amount of time required for a full forced expiration, the presence of acute bronchoconstrictive disease tends to decrease the peak expiratory flow measured over a typical forced exhalation.

The term “porous material” shall be interpreted to mean a material having any shape such as the shape of a sheet or plate having any given outer perimeter shape, but preferably covering a package opening wherein the material has a plurality of openings therein, which openings may be placed in a regular or irregular pattern, and which openings preferably have a diameter in the range of 0.25 micron to 6 microns and a pore density in the range of 1×10⁴ to about 1×10⁸ pores per square centimeter. Alternatively, the porous material may be merely an area of the package which has porous position therein wherein the pores have a size and a density as described above. The configuration and arrangement of the pore density may be changed so as to provide pores which are capable of creating an aerosol. For example, the porous material or area of the container may have some 10 to 10,000 pores therein which pores are positioned in an area of from about 1 sq. mm. to about 1 sq. cm. The material is preferably comprised of a material having a density in the range of 0.25 to 3.0 mg/cm², more preferably 1.7 mg/cm², and a thickness of about 2 to about 20 microns, more preferably 8 to 12 microns. The material is preferably hydrophobic and includes materials such as polycarbonates and polyesters which may have the pores formed therein by any suitable method including anisotropic etching or by etching through a thin film of metal or other suitable material. Pores can be created in the material which may be an area of the container by use of techniques such as etching, plating or laser drilling. The materials may have pores with a conical configuration and have sufficient structural integrity so that it is maintained intact (will not rupture) when subjected to force in the amount of about 20 to 1000 psi while the formulation is forced through the pores. The porous material functions to form an aerosolized mist when the formulation is forced through it. Those skilled in the art may contemplate other materials which achieve this function as such materials are intended to be encompassed by this invention.

The term “nozzle” includes a “porous member” and any opening or group of openings in a single dose container which is used to aerosolize the contents of the container. The nozzle may be comprised of an opening which allow for the creation of converging jets that collide and breakup to form respirable particles such as the Boehringer-Ingelheim Respimat. The nozzle or porous member may be comprised of any suitable material or combination of materials.

The terms “aerosol”, “aerosolized formulation” and the like are used interchangeably herein to refer to a volume of air which has suspended within it particles of a formulation comprising a drug or diagnostic agent wherein the particles have a diameter in the range of 0.5 to 12 microns, for respiratory therapy, or in the range of 15 to 50 microns for ocular therapy.

The terms “individual”, “subject” or “patient” used interchangeably herein refer to a mammal, generally a human.

The term “particle size” refers to the aerodynamic diameter of the particles. The aerodynamic diameter is defined as the diameter of a particle with a unit density of (1 g/cm³) which has the same terminal sedimenation velocity in air as the particle in question.

General Description

This invention provides a system for generating an aerosol comprising (a) a multiple-dose container of a sterile drug solution, or sterile drug suspension, or sterile drug powder to be reconstituted into a sterile liquid preparation for aerosolization with sterile liquid such as USP Water for Inhalation, sterile saline or the like, (b) a device which provides the means for generation and delivery of an aerosol, and (c) a disposable package which can be loaded into the device and filled with drug or liquid for drug reconstitution from the multiple-dose container and which is further comprised of a single-dose container with a means of drug intake. The single-dose container may include an exit opening or a nozzle which may be a porous material and/or an area of pores or nozzles therein, operably associated with the single-dose container. The single dose container does not need to contain the nozzle for aerosol generation but may be instead connected to such a means of aerosol generation, preferable after it has been disconnected from the multiple dose container to avoid the contamination of the latter. The invention may further comprise a formulation transfer member to transfer the sterile drug from the multiple-dose container to the single dose container without breaching the sterile integrity of the remaining contents of the multiple-dose container.

The present invention also pertains to the processes for aerosol generation from a sterile formulation and delivery generally comprising the steps of intaking a pharmaceutically active drug or liquid for drug reconstitution from a multiple-dose container into a single-dose container of a disposable package. From the single-dose container the contents are aerosolized, e.g., by forcing or otherwise passing the pharmaceutically active drug through a a nozzle, such as a plurality of pores operably associated with the single-dose container and/or by directly or indirectly aerosolizing the drug, for example by vibrating the drug solution with a piezoelectric system to aerosolize it and deliver the aerosol to a patient.

Disposable Package

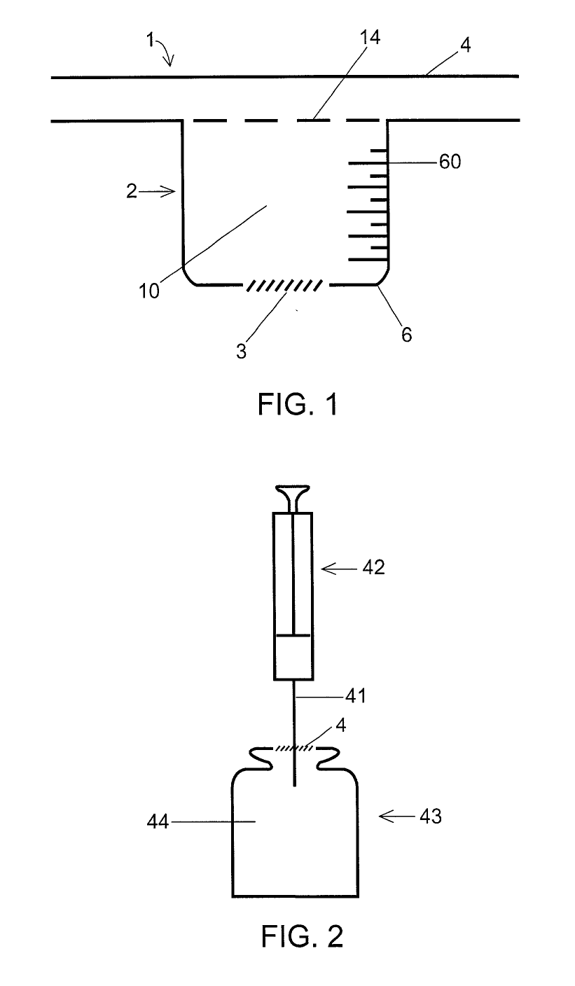

FIG. 1 shows an embodiment of the present invention comprising a disposable package 1. The disposable package 1 is comprised of at least a single-dose container 2 with a means for drug intake such as a self-sealing, re-sealable area 3 and a nozzle such as an array of orifices or channels or a porous material 14 and/or an area of pores therein, operably associated with the single-dose container 2. The single-dose container may further include indices 60 thereon in the form of visually (or machine) readable numbers, letters or other indices which can be readily perceived by the user (or delivery device) for content level determination and /or a removable cover 4 positioned over at least a portion of the nozzle porous material 14 in order to prevent contamination of the contents of the single-dose container 2.

The single-dose container 2 enables sterile drug or fluid for drug reconstitution from a multiple-dose container to be transferred to the disposable package 1 such that the sterile drug preparation can be retained within the single-dose container 2 of the disposable package 1 before it is aerosolized, e.g., transferred drug or reconstituted drug 10 of the figure. When drug is to be reconstituted inside the single-dose container 2, the single-dose container 2 can be pre-filled with a solid drug (e.g., prepared by sterile freeze-drying, etc.), for example a drug unstable in liquid form which could be reconstituted prior to use by the transfer of an appropriate sterile liquid such as USP Water for Inhalation, sterile saline, or the like. As such, the single-dose container 2 of the disposable package 1 is comprised of a means to intake drug or fluid from a multiple-dose container (e.g., the re-sealable area 3 of the figure or tubular member 5 of FIG. 4), whereby the sterile drug or liquid is effectively transferred and retained inside the single-dose container 2 of the disposable package 1. The single-dose container 2 can generally be described as being further comprised of an entrance side comprising the means for the intake of a drug or liquid for drug reconstitution from a multiple-dose container and an exit side from which a pharmaceutically active drug is released. In certain embodiments, at least one wall of the single-dose container is collapsible by the application of a force.

The entrance side, e.g., a wall 6 of the single-dose container provides the means for the intake of sterile drug or sterile liquid for drug reconstitution into the single-dose container. In the embodiment of FIG. 1, sterile drug or liquid for drug reconstitution is transferred from a multiple-dose container into the single-dose container 2 via a separate formulation transfer member such as an interconnecting needle, e.g., a needle of a syringe or the like. Thus, according to this embodiment, both the single-dose container and the multiple-dose container are comprised of a self-sealing, re-sealable area penetrable by the formulation transfer member. In certain embodiments, the self-sealing, re-sealable area on one or both containers is comprised of a bactericide or a general microbicidal agent, e.g., a bactericide coating on all or parts of the self-sealing, re-sealable area or a sponge coated or soaked with a bactericide operably associated with the area. Exemplary self-sealing, re-sealable areas may comprise a rubber stopper or the like whereby the areas re-seal after being penetrated, for example when punctured by a needle of any gauge and preferably of a gauge useful for injecting drugs into humans e.g. about 20 gauge or less, a re-sealable septum, a valve or a passageway and/or are substantially similar to those which are described in U.S. Pat. Nos.: 5,971,181, 5,921,419, 5,006,113 and 5,423,791, herein incorporated by reference. It will be appreciated that any other self-sealing, re-sealable areas of this or a similar kind may be employed as well.

In the embodiment of FIG. 2, the external surface of a self-sealing, re-sealable area 40 of a multiple-dose container 43 is sterilized, e.g., with an alcohol swab, or other type of microbicidal agent or another antiseptic solution, or is sterilized by some other means (radiation, heat, etc.), immediately prior to use. A needle 41 of a hypodermic syringe 42 is pushed through a self-sealing, re-sealable area 40 of a multiple-dose container 43. The needle 41 makes contact with the sterile solution, suspension or fluid for drug reconstitution 44 in the multiple-dose container 43 and the sterile drug or fluid 44 in the multiple-dose container is then transferred through the needle 41 into the syringe 42 and then through the self-sealing, re-sealable area 3 of the entrance side 6 of the single-dose container 2. Once the needle 41 is withdrawn from the multiple-dose container 43 after the transfer of the sterile contents into the single-dose container 2, the self-sealing, re-sealable area 40 on the multiple-dose container 43 re-seals, thus maintaining the sterility of the remaining drug doses therein. Thus, the invention enables repeated transfer of sterile drug or liquid from a multiple-dose container into a single-dose container for subsequent aerosolization, while maintaining the sterility of the remaining contents of the multiple-dose container.

Once transfer of sterile drug or fluid for reconstitution is complete, a tear-away top or cover 4, if present, can be removed and the formulation 10 inside the single-dose container 2 can be forced or otherwise passed outward through the aerosol forming nozzle which could be a material with holes in it, or a porous material as represented by 14, creating an aerosol for example by passing the drug solution through it under pressure, and/or with the aid of vibration.. In many embodiments, the single-dose container 2 has at least one collapsible cylindrical wall with bellows or accordion-like undulations as illustrated in FIG. 6, so that the bottom of the single-dose container 2 can be forced upwards towards the top of the single-dose container 2 by a piston or the like (not shown) to allow the contents 10 in the single-dose container 2 to be forced out of the single-dose container, through the a nozzle, for examples shown as a plurality of pores in the porous material 14.

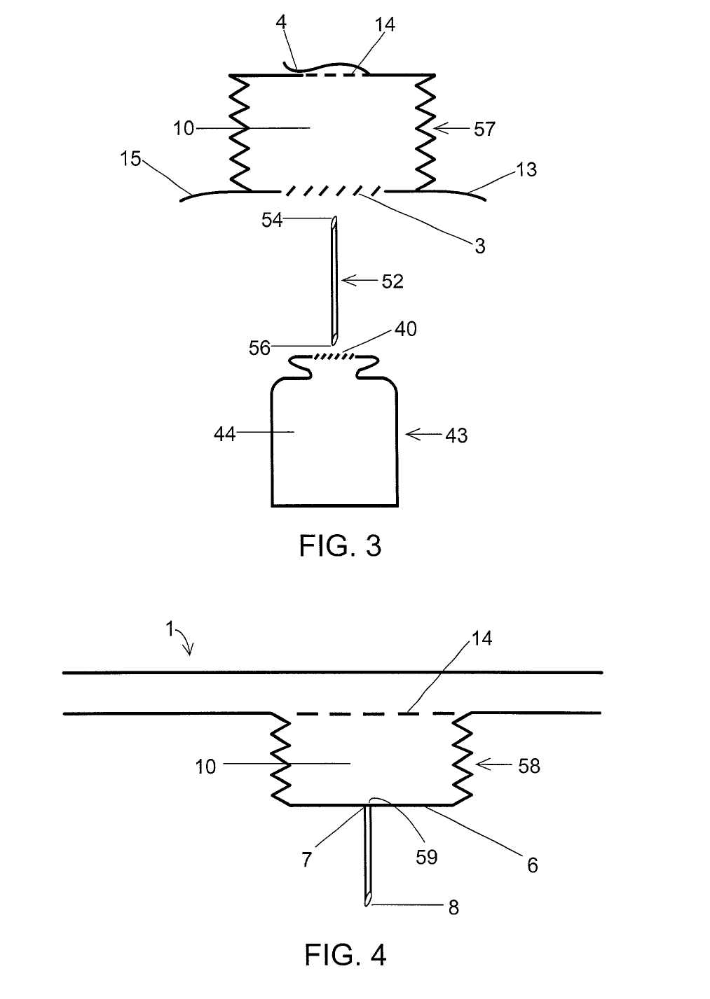

In another embodiment as shown in FIG. 3, the formulation transfer member is an interconnecting needle 52, preferably a needle of 20 gauge or less, which can be removed from the re-sealable area 3 after sterile drug or liquid transfer thereto for aerosolization or can remain attached to the single-dose container for injection into the patient via the same interconnecting hollow needle 52. The embodiment of FIG. 3 shows the interconnecting needle 52 with substantially pointed ends 54 and 56 and a passageway therethrough. In this embodiment, the needle 52 is sterilized and then pushed through both the self-sealing, re-sealable area 3 of the single-dose container 57 and the self-sealing, re-sealable area 40 of the multiple-dose container 43 (FIG. 2) and sterile drug or fluid is transferred into the single-dose container 57 from the multiple-dose container 43. In one example, the transfer can be effectuated by grasping the finger grips 13 and 15 and pushing downward to collapse the single-dose container 57, creating a negative pressure to draw fluid up upon re-expansion. After transfer, the needle 52 can then be removed from both the single-dose container 57 and multiple-dose container 43. If aerosolization is the preferred mode of delivery, the optional tear-away. away top or removable cover 4, can be removed from the single-dose container 57. If present, the tear-away top or cover 4 is positioned over at least a portion of the nozzle e.g. porous material 14 in order to prevent contamination of the formulation 10. After the removal of the tear-away top or cover 4, the formulation in the single-dose container can then be forced or otherwise passed outward through the porous material 14 operably associated with the single-dose container. Alternatively, if injection is the preferred mode of delivery, the needle 52 can be removed from the multiple-dose container 43 while remaining attached to the single-dose container 57. End 56 is placed in contact with the target injection site of the patient and force can be applied to the single-dose container 57 by grasping the finger grips 13 and 15 and pushing downward to collapse at least one wall of the single-dose container 57, forcing the contents out through the attached needle 52 for injection into the patient.

FIG. 4 shows another embodiment of the invention where the means for drug intake is a conduit, e.g., a tubular member 5 extending through the entrance side, i.e., a wall 6 of the single-dose container 58. As such, the tubular member 5 is at least comprised of a proximal end 7, a distal end 8 and an open channel, e.g., a needle. The tubular member 5 may be attached to the single-dose container wall 6 by any suitable means including glue, polymeric bonding, sealing materials, and the like. The tubular member 5 may be made from any suitable material including metals, alloys and various polymeric compounds. In those embodiments where the tubular member 5 is a needle, the needle may be made from a metal such as stainless steel, metal alloy or any such metal as is suitable for the intended purpose and known to those of skill in the art. In certain embodiments, the single-dose container 58 is further comprised of a breakable seal 59 , which maintains reduced pressure in the single-dose container 58 until the needle 5 is placed in a liquid formulation and the seal is broken, allowing the pressure to move into the reduced pressure of the single-dose container 58.

In the embodiment of FIG. 4 the external surface of a self-sealing, re-sealable area of a multiple-dose container is sterilized, e.g., with an alcohol swab, or some other form of microbicidal agent, or another antiseptic solution, or is sterilized by some other means (radiation, heat, etc.), immediately prior to use. In many embodiments, the self-sealing, re-sealable area of a multiple-dose container is further comprised of a microbicidal agent, e.g., a bactericide coating or sponge or the like comprised of a bactericide, such that the needle 5 is initially pushed through the sponge before entering the multi-dose container.

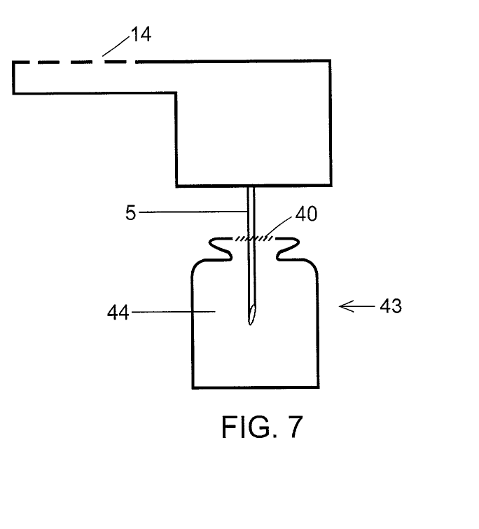

In operation, the needle 5 is pushed through a self-sealing, re-sealable area 40 of a multiple-dose container 43, as shown in FIG. 7. The needle 5 makes contact with the sterile solution, suspension or fluid for drug reconstitution 44 in the multiple-dose container 43. Such transfer can be accomplished by pressure, for example positive pressure may be applied to the multiple-dose container 43, or negative pressure may be applied to the single-dose container 59, to transfer the desired aliquot of sterile drug or fluid thereto. The pressure may be provided manually, electrically (e.g., by the force of an electrically driven piston) or using stored energy (e.g., by a evaporative expansion of a low boiling point propellant, or by movement of a piston driven by the energy stored in a compressed spring). Employing pressure to assist in effectuating the transfer enables withdrawal of the needle from the self-sealing, re-sealable area of the multiple-dose container while still preserving the sterility of the contents of this container. Also, by maintaining a higher pressure in the multiple-dose container 43 than in the single-dose container 58 such that no transport of any microorganisms into the multiple-dose container can take place, aerosol generation may be performed while the single-dose container 58 and the multiple-dose container 43 are still connected, yet the sterile integrity of the remaining contents of the multiple-dose container is maintained. If the needle 41 (see FIG. 2) is withdrawn from the multiple-dose container 43 after the transfer of the sterile contents into the single-dose container 58, the self-sealing, re-sealable area 40 on the multiple-dose container 43 re-seals, thus maintaining the sterility of the remaining drug doses therein. Thus, the invention enables repeated transfer of sterile drug from a multiple-dose container 43 into a single-dose container (2, 57, or 58 etc.) for subsequent aerosolization, while maintaining the sterility of the remaining drug doses in the multiple-dose container.

Once transfer of sterile drug or fluid for reconstitution is complete, the tear-away top or cover 4, if present, can be removed and the formulation 10 inside the single-dose container 60 can be forced or otherwise passed outward through any nozzle such as the porous material 14, creating an aerosol. In many embodiments, the single-dose container 60 (see FIG. 6) has at least one collapsible cylindrical wall with bellows or accordion-like undulations as illustrated in FIG. 6, so that the bottom of the single-dose container 60 can be forced upwards, by means of a piston or the like (not shown) towards the top of the single-dose container to allow the contents in the single-dose container 60 to be forced out of the single-dose container, through the plurality of pores in the porous material 14.

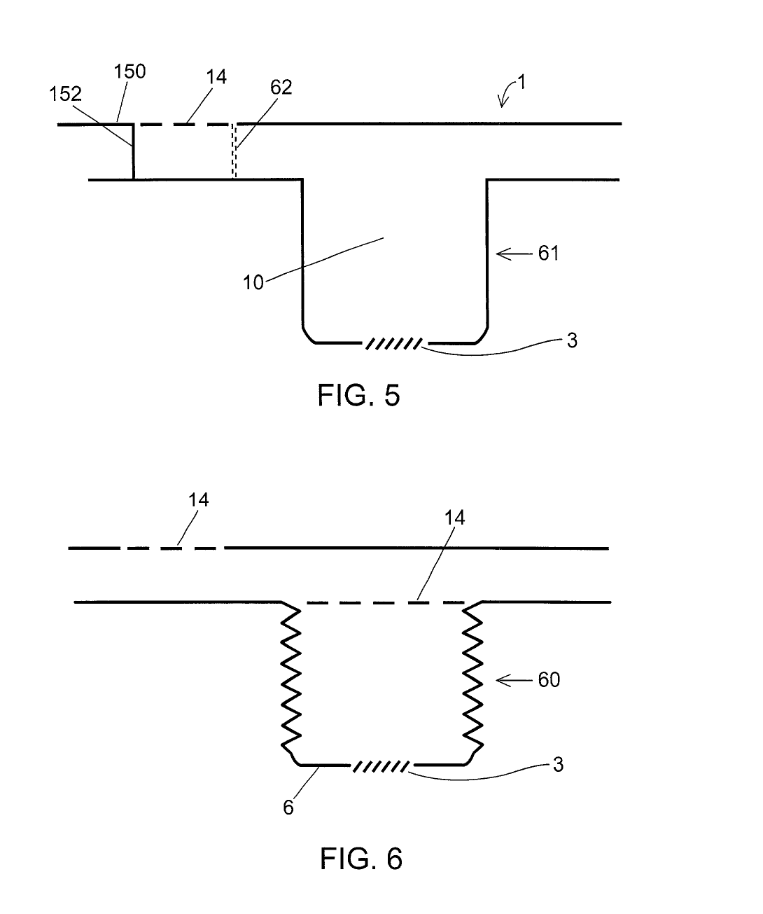

Regardless of the means for formulation or fluid for drug reconstitution transfer, aerosolization of the sterile drug formulation retained inside the single-dose container is effectuated by forcing or otherwise passing the formulation through a means of aerosol generation, such as a nozzle which could be in the form of pores operably associated with the single-dose container, e.g., the porous material 14 in FIG. 1, as described above. The pores of the invention can either be an area of pores in the single-dose container 2 or a porous material operably associated with the single-dose container 2. Thus, in one example, the porous material 14 covers an opening in the single-dose container 2. The aerosolizing orifice such as a porous material 14 may be directly above the single-dose container 2 as shown in FIG. 1, or may be located at a distance from the single-dose container 61 as shown in FIG. 5, whereby the sterile drug is forced or otherwise passed out of the single-dose container, to travel through a channel and then through the nozzle which in this case is a porous material. In the embodiment of FIG. 5, the porous material 14 is a part of a solid cover 150. In this embodiments where at least one wall of a single-dose container is collapsed to force the formulation 10 out, the formulation 10 is forced against a barrier 62 which is broken upon the application of a force causing a pressure of 50 psi or less. The formulation 10 flows through the channel 11 until it is stopped by the abutment 152, after which pressure builds within the channel 11 and the formulation 10 is forced outward through the porous material 14.

Regardless of the location of the aerosol generating nozzle 14, it is preferably dry prior to use. Further, a nozzle material for aerosol creation according to the present invention provides relatively small particle sizes for delivery to the respiratory tract (aerodynamic diameter less than about 5 micrometers), or larger particles for ocular delivery. The system can aerosolize from about 5 μl to about 300 μl, more preferably, 50 μl of liquid from one single-dose container for pulmonary delivery. The contents of a single-dose container is generally aerosolized in a relatively short period of time, e.g., 1 second or less and inhaled by the patient in a single breath or delivered ocularly in a similarly protracted period of time.

The material of the present invention can be produced wherein the openings or pores are all uniform in size and are positioned at uniform distances from each other. However, the openings can be varied in size and placed in various patterns or randomly on the material. If the size of the openings is varied the size of the particles formed will also vary. In general, it is preferable to maintain uniform opening sizes in order to create uniform particle sizes and it is particularly preferable to have the opening sizes within the range of about 0.25 to about 6 microns which will create particle sizes of about 0.5 to 12 microns which are preferred with respect to inhalation applications. When the openings have a pore size in the range of 0.5 to 1.5 microns they will produce an aerosol having particle sizes in the range of 1 to 3 microns which is particularly useful for treating the bronchioles and alveoli. Pore sizes having a diameter of about 1.5 to 2.5 microns will produce particle sizes having a diameter of about 3 to 5 microns which are particularly useful with respect to treating the bronchi.

Although the pores are generally smaller, the present invention includes a porous material with pore sizes in the range of 0.5 micron to about 50 microns. Further, the pores are preferably separated, one from the other, providing about 1×10⁴ to about 1×10⁸ pores/cm². The material may include from 10 to 10,000 pores over an area of from 1 sq. mm. to 1 sq. cm. Further, the pore diameter indicates that at least 75% of the pores on the material fall within the prescribed range and preferably indicates that 85% or more of the pores fit within the prescribed range. Uniformity in pore size is desirable for creating uniformity in the particle size of the aerosol being delivered which is important with respect to maintaining consistency in dosing.

A variety of different types of materials can be used for forming the aerosol generating nozzles. It is important that the material material which the nozzle is placed in has sufficient structural integrity such that when the liquid in the container is being aerosolized with the aid of the material, the material will not rupture and the nozzle hole size will remain essentially constant under pressure. Examples of such materials will be apparent to one of skill in the art upon reading this disclosure.

Although the thickness of the material may be of any thickness, it is desirable for the material to be particularly thin, e.g. less than one millimeter and more preferably less than 20 μm with particularly preferred components having a thickness in the range of about 10 μm to 15 μm. As the thickness of this material is increased the amount of energy necessary to force the liquid through the material is increased. Since the device of the present invention is a hand-held device it is important to produce materials which require the use of small amounts of energy in order to create the aerosol in that the energy supply is somewhat limited.

In order to reduce the amount of energy needed to force the formulation through the pores of the porous material it is possible to produce the pores in different configurations. Although the pores are generally cylindrical in shape they can be non-cylindrical (e.g. hourglass shaped) and are preferably conically shaped. The surface of the conically shaped pores may have a coating thereon of a reduced friction type of material such as Teflon-type materials.

Device for Aerosol Generation and Delivery

The single-dose container of the present invention can be loaded into a range of different types of devices for aerosol generation and delivery, i.e., a drug-dispensing or aerosolizing device (“device”). Examples of drug-dispensing devices operable with the present invention are ones which are the same as or substantially similar to the devices disclosed in U.S. Pat. No.: 5,544,646, herein incorporated by reference. However, other devices utilizing other modes of aerosol generation may also be used with the present invention and will be apparent to one of skill in the art upon reading this disclosure. For example, air jet nebulizers and ultrasonic nebulizers are also suitable for use with the present invention. In general, the drug dispensing device is a hand-held, portable device for holding a disposable package with at least one single-dose container, and may further comprise a mechanical mechanism for forcing the contents of a singe-dose container of a disposable package through a nozzle such as a porous material operably associated with the single-dose container or a vibrating means directly or indirectly associated with the contents, and a mouthpiece to deliver the aerosolized drug to the patient. The device may be a large aerosol generator which is not hand-held but rather a “table-top” device. The device may also be a part of a ventilation apparatus used in critical care units. The sterile nature of the aerosol could be particularly advantageous for use in people at risk in intensive care units as these patients could develop serious nosocomial infections if the solutions are not sterile.

The single-dose container can be loaded into the drug-dispensing device. Examples of drug-dispensing devices operable with the present invention are ones which are the same as or similar to the devices disclosed in U.S. Pat. Nos.: 5,544,646, 5,662,271 and 5,497,944, herein incorporated by reference. However, other devices utilizing other modes of aerosol generation may also be used with the present invention and will be apparent to one of skill in the art upon reading this disclosure. For example, it will be appreciated that devices such as air jet nebulizers, ultrasonic nebulizers and other piezoelectric aerosol generation systems, or other devices of these or a similar kind could be employed with the present invention. In one embodiment of the invention, the single-dose container includes at least one wall which can be collapsed to allow the liquid contents to be forced out of the single-dose container and through the porous material or the area of pores therein, as described above. Thus, once the single-dose container has been loaded with drug, it is loaded into an appropriate drug dispensing device and at least one wall is collapsed by the application of force, e.g., a force causing a pressure of about 20 psi to 1000 psi, provided by the drug-dispensing device. The force can be actuated by the patient via a trigger-like mechanism on the device or by breath actuation, as is commonly known to one of skill in the art. Such force can be provided by a mechanical mechanism of the drug-dispensing device. In exemplary embodiments, pressure is applied to the single-dose container such as by the force provided from a mechanical mechanism, e.g., a piston. After the force is applied, the single-dose container is collapsed and the sterile drug within the single-dose container is forced out, in certain embodiments through a channel. The single-dose container has at least one collapsible cylindrical wall with bellows or accordion-like undulations so that the bottom of the single-dose container can be forced upwards towards the top of the single-dose container and allow the contents in the single-dose container to be forced out of the single-dose container, through a nozzle or a plurality of pores to create an aerosol.

In yet another embodiment, the creation of the small particles may be facilitated by the use of a vibrating means directly or indirectly associated with the sterile solution to be aerosolized. For example, a vibrating means could directly vibrate the sterile solution whilst in the single-dose container or it may vibrate an orifice, porous area, porous material or the like through which the sterile solution passes. The vibrating means is designed so as to generate vibrations which affect the particle formation of formulation being forced out of the pores. In one embodiment, the vibrating means provides a vibration frequency in the range of about 800 to about 4000 kHz. Those skilled in the art will recognize that some adjustments can be made in the pore size, vibration frequency, pressure, and other parameters based on the density and viscosity of the formulation keeping in mind that the object is to provide aerosolized particles having a diameter in the range of about 0.5 to 12 microns for inhalation delivery, or larger particles for ocular delivery. Accordingly, a vibrating means can be used alone to generate an aerosol or can be used in conjunction with a mechanical mechanism such as a piston or the like to force the formulation through a nozzle or pores. (Devices of use with the present invention include, for example, ultrasonic nebulizers using a piezoelectric crystal to produce liquid droplets, devices which use an ultrasonic vibrator to vibrate a nozzle, perforated membrane or the like and vibrating means with a mechanical mechanism. (See U.S. Pat. Nos.: 5,261,601, 4,533,082, 5,758,637, 5,515,841, 5,586,550, 5,518,179, and 5,544,646, the disclosures of which are herein incorporated by reference.))

The single-dose container loaded into the dispensing device can be in any desired size. In most cases, the size of the single-dose container is not directly related to the amount of drug being delivered in that most formulations include relatively large amounts of excipient material e.g. water or a saline solution. Accordingly, a given size container could include a wide range of different doses by varying drug concentration. The amount of drug delivered to the patient will vary greatly depending on the particular drug being delivered. In accordance with the present invention it is possible to deliver a wide range of different drugs.

The mouthpiece of the dispensing device provides a conduit through which the aerosolized drug is delivered to the patient. As such, the mouthpiece may be permanently or removabley attached and may be affixed in place or rotatable.

Multiple-Dose Container

Prior to being transferred to the single-dose container of the present invention, sterile drug formulation or sterile liquid for drug reconstitution is retained in a multiple-dose container 43, shown in FIG. 2. The multiple-dose container is comprised of a self-sealing, re-sealable area 40 as described above, such that a needle or the like may be pushed through the area 40 to the contents 44 of the multiple-dose container and then removed and the area 40 re-seals around the entry point of the needle to maintain the sterile integrity of the remaining contents 44 in the multiple-dose container. The area 40 may further comprise a microbicidal agent or the like, for example the area 40 or portions of the area 40 may be coated with a bactericide or a sponge comprised of a bactericide may be operably associated with the area 40 such that a needle 40 or the like is placed in contact with the bactericide before entering the contents of the multi-dose container 43. Exemplary self-sealing, re-sealable areas may comprise a rubber stopper, septum, valve or passageway or the like and/or are similar to those which are described in U.S. Pat. Nos.: 5,971,181, 5,921,419, 5,006,113 and 5,423,791, herein incorporated by reference. It will be appreciated that any other multiple-dose container of these or similar kinds could be employed with the present invention.

The multiple dose container may hold drugs or drug formulations or may hold a sterile liquid such as USP Water for Inhalation, sterile saline or the like for reconstitution of a solid drug retained in a single-dose container. Formulation within the multiple-dose container may comprise drugs that are unstable in liquid form and thus could be kept as solids (e.g., prepared by sterile freeze-drying) in the multiple-dose container. A sterile liquid such as USP Water for Inhalation, sterile saline for inhalation, etc., could be introduced into the multiple-dose container to dissolve the solid prior to use.

The drug formulation is preferably in a low viscosity liquid formulation which is most preferably a formulation which can be aerosolized easily and includes respiratory drug formulations currently used in nebulizers. Additionally, pharmaceutically active drugs for ocular therapies are also of use in the invention. The viscosity of the drug by itself or in combination with a carrier must be sufficiently low so that the formulation can be forced through the porous material to form an aerosol, e.g., using a force in the range from about 20 to 1000 psi to form an aerosol preferably having a particle size in the range of about 0.5 to 12 microns for pulmonary delivery or larger for ocular delivery.

For example, the drugs transferred from the multiple-dose container to single-dose container could be drugs which have a systemic effect such as narcotic drugs, for example fentanyl, sufentanil, or anxiolytic drugs such as diazepam midazolam as well as peptide drugs, e.g., proteins, antibodies, soluble receptors, insulin and its analogs, calcitonin and other peptides, nucleotide sequences, gene vectors, vaccines and other therapeutic and diagnostic agents delivered to the respiratory tract for the prevention or treatment of respiratory disease, or therapeutic and prophylactic activity outside the respiratory tract.. In addition, mixed agonist/antagonist drugs such as butorphanol can also be used for the management of pain delivered to provide relief from pain or anxiety. The preferred respiratory drugs are bronchodilators such salmeterol, terbutaline, albuterol, anti-inflammatory drugs such beclomethasone dipropionate, triamcinolone acetonide, flunisolide, prophylactic drugs such as cromolyn sodium or nedocromil, ipratropium bromide, antibiotics, recombinant human rhDNase and include, free acids, bases, salts and various hydrate forms thereof.

FIG. 7 shows a single dose container 63 with a hollow table 5 (as per FIG. 4) attached thereto. The table 5 is inserted into a multi-dose container 43 which is holding formulation 44 under pressure. The pressure differential between the inside of container 43 and single dose container 63 causes the formulation 44 to enter the container 63. The container 63 can then be loaded into a device where its contents can be moved out of the nozzle 14 to create an aerosol.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method for delivering an aerosolized flowable liquid formulation into a patient, comprising:
(a) aseptically intaking a pharmaceutically active drug from a multiple-dose container into a single dose container such that contents of the multiple dose container do not become contaminated with microorganism;
(b) moving said pharmaceutically active drug out of the single-dose container; and
(c) aerosolizing said pharmaceutically active drug that has been transferred into the single dose container;
whereby said aerosolized drug is delivered to a patient.

2. The method according to claim 1, wherein said intaking is accomplished through a self-sealing, re-sealable area on a wall of said single dose container.

3. The method according to claim 2, wherein said self-sealing, re-sealable area is a rubber stopper, septum, valve or passageway.

4. The method according to claim 2, wherein said intaking is further comprised of transferring said pharmaceutically active drug via a formulation transfer member with an open channel, through said re-sealable area on said wall, whereby said tubular member punctures a self-sealing re-sealable area of said multiple-dose container to withdraw an aliquot of sterile drug formulation before transferring said formulation to said single-dose container.

5. The method according to claim 4, wherein said tubular member is a needle.

6. The method according to claim 5, wherein said needle is connected to a syringe.

7. The method according to claim 1, wherein said intaking is accomplished through a tubular member extending through a wall of said single dose container, said tubular member comprising an open channel.

8. The method according to claim 7, wherein said tubular member is a needle.

9. The method according to claim 7, wherein said tubular member punctures a self-sealing, re-sealable area on said multiple-dose container to withdraw an aliquot of sterile drug formulation therein.

10. The method of claim 1, wherein said moving is accomplished by collapsing a wall of the single-dose container and forcing the contents out.

11. The method according to claim 10, wherein said forcing is accomplished by a mechanical mechanism.

12. The method according to claim 11, wherein said mechanical mechanism is a piston.

13. The method according to claim 1, wherein said aerosolizing is accomplished by forcing or otherwise passing said pharmaceutically active drug through a nozzle.

14. The method according to claim 13, wherein said nozzle is a portion of said single-dose container.

15. The method according to claim 13, wherein said nozzle is a porous material.

16. The method according to claim 1, further comprised of vibrating said pharmaceutically active drug to aerosolize it.

17. The method of claim 16, wherein said vibrating is accomplished by a vibrating means capable of vibrating at a frequency in the range of 800 to 4,000 kilohertz.

18. A single-dose container, comprising:
(a) a wall which holds fluid therein;
(b) a nozzle which aerosolizes the fluid when fluid is moved through the nozzle,
(c) a tubular member extending through a wall of said single-dose container, the tubular member comprising an open channel.

19. The single-dose container of claim 18, wherein said wall is collapsible upon the application of force.

20. The single-dose container of claim 19, wherein said application of force is to a bottom wall so as to collapse each side wall in a manner so as to force essentially all of said fluid out of said single-dose container and through said nozzle to create an aerosol.

21. The single-dose container according to claim 19, wherein said nozzle is comprised of a porous material with a pore density of from about 1×10⁴ through about 1×10⁸ pores/cm² and the wall is collapsed by said application of force causing a pressure of about 20 psi to 100 psi.

22. The single-dose container of claim 18, further comprised of a vibrating means.

23. The single-dose container of claim 21, wherein said vibrating means is capable of vibrating at a frequency in the range of 800 to 4,000 kilohertz.

24. The single-dose container according to claim 18, wherein said fluid is a pharmaceutically active drug.

25. The single-dose container of claim 24, wherein said pharmaceutically active drug is a respiratory drug.

26. The single-dose container of claim 24, wherein said pharmaceutically active drug is a systemically acting drug.

27. The single dose container of claim 24, wherein said pharmaceutically active drug is a steroid selected from the group consisting of beclomethasone, dipropionate, flunisolide, fluticasone and triamcinolone acetonide.

28. The single-dose container of claim 24, wherein said pharmaceutically active drug is a non-steroidal, anti-inflammatory drug.

29. The single-dose container of claim 24, wherein the pharmaceutically active drug is selected from the group consisting of isoproterenol, antibiotics, recombinant human rhDNase, cromolyn sodium, albuterol sulfate, metaproterenol sulfate, salmeterol xinaforate and formotorol.

30. The single-dose container according to claim 1, wherein said pores are present in said porous material with a pore density of from about 1×10⁴ through about 1×10⁸ pores/cm² and the wall is collapsed by said application of force causing a pressure of about 20 psi to 100 psi.

31. The single-dose container according to claim 18, wherein said tubular member is a needle.

32. The single-dose container according to claim 31, wherein said needle is about 20 gauge or less.

33. A single-dose container, comprising:
(a) a wall which holds fluid;
(b) a porous material covering an opening in the container, the porous material comprising pores having a diameter in the range of from about 0.25 micron to about 6.0 microns; and
(c) a self sealing, re-sealable area on said wall of said single-dose container, said self sealing, re-sealable area being characterized by a self-sealing ability when punctured by a needle of about 20 gauge or less.

34. The single-dose container of claim 33, wherein said wall is collapsible upon the application of force.

35. The single-dose container of claim 34, wherein application of force is to a bottom wall so as to collapse each side wall in a manner so as to force essentially all of said fluid out of said single-dose container and through said porous material to create an aerosol.

36. The single-dose container according to claim 34, wherein said porous material has a pore density of from about 1×10⁴ through about 1×10⁸ pores/cm² and the wall is collapsed by said application of force causing a pressure of about 20 psi to 100 psi.

37. The single-dose container of claim 33, further comprised of a vibrating means.

38. The single-dose container of claim 33, wherein said vibrating means is capable of vibrating at a frequency in the range of 800 to 4,000 kilohertz.

39. The single-dose container according to claim 33, wherein said fluid is a pharmaceutically active drug.

40. The single-dose container of claim 39, wherein said pharmaceutically active drug is a respiratory drug.

41. The single-dose container of claim 39, wherein said pharmaceutically active drug is a systemically acting drug.

42. The single dose container of claim 39, wherein said pharmaceutically active drug is a steroid selected from the group consisting of beclomethasone, dipropionate, flunisolide, fluticasone and triamcinolone acetonide.

43. The single-dose container of claim 39, wherein said pharmaceutically active drug is a non-steroidal, anti-inflammatory drug.

44. The single-dose container of claim 39, wherein the pharmaceutically active drug is selected from the group consisting of isoproterenol, antibiotics, recombinant human rhDNase, cromolyn sodium, albuterol sulfate, metaproterenol sulfate, salmeterol xinaforate and formotorol.

45. The single-dose container according to claim 33, wherein said self-sealing, re-sealable area is a rubber stopper, septum, valve or passageway.

46. The single-dose container according to claim 33, wherein said porous material has a pore density of from about 1×10⁴ through about 1×10⁸ pores/cm² and the wall is collapsed by said application of force causing a pressure of about 20 psi to 100 psi.

47. A method for delivering an aerosolized flowable liquid formulation into a patient, comprising:
(a) intaking a pharmaceutically active drug from a multiple-dose container into a single dose container,
(b) aerosolizing said pharmaceutically active drug, and
(c) moving said pharmaceutically active drug out of the single-dose container, whereby said aerosolized drug is delivered to a patient.

48. The method according to claim 21, wherein said intaking is accomplished through a self-sealing, re-sealable area on a wall of said single dose container.

49. The method according to claim 22, wherein said self-sealing, re-sealable area is a rubber stopper, septum, valve or passageway.

50. The method according to claim 22, wherein said intaking is further comprised of transferring said pharmaceutically active drug via a formulation transfer member with an open channel, through said re-sealable area on said wall, whereby said tubular member punctures a self-sealing re-sealable area of said multiple-dose container to withdraw an aliquot of sterile drug formulation before transferring said formulation to said single-dose container.

51. The method according to claim 24, wherein said tubular member is a needle.

52. The method according to claim 25, wherein said needle is connected to a syringe.

53. The method according to claim 21, wherein said intaking is accomplished through a tubular member extending through a wall of said single dose container, said tubular member comprising an open channel.

54. The method according to claim 27, wherein said tubular member is a needle.

55. The method according to claim 27, wherein said tubular member punctures a self-sealing, re-sealable area on said multiple-dose container to withdraw an aliquot of sterile drug formulation therein.

56. The method according to claim 21, wherein said forcing is accomplished by a mechanical mechanism.

57. The method according to claim 30, wherein said mechanical mechanism is a piston.

58. The method according to claim 21, wherein said aerosolizing is accomplished by forcing said pharmaceutically active drug through pores.

59. The method according to claim 32, wherein said pores are in a portion of said single-dose container.

60. The method according to claim 32, wherein said pores are on a porous material.

61. The method according to claim 21, further comprised of vibrating said pharmaceutically active drug with a vibration device capable of vibrating at a frequency in the range of 800 to 4,000 kilohertz.
